(19) United States
(12) Patent Application Publication
(10) Pub. No.: US 2026/0047780 A1
(43) Pub. Date: Feb. 19, 2026

(54) WEARABLE TRANSPLANTABLE TISSUE VIABILITY BIOSENSOR

(71) Applicant: The Regents of the University of California, Oakland, CA (US)

(72) Inventors: Jesse R. Qualliotine, San Diego, CA (US); Joseph Wang, San Diego, CA (US)

(21) Appl. No.: 18/807,831

(22) Filed: Aug. 16, 2024

(51) Int. Cl. A61B 5/145; A61B 5/00; A61B 5/1473; A61B 17/00

(52) U.S. Cl. CPC A61B 5/14514; A61B 5/14532; A61B 5/14546; A61B 5/14735; A61B 5/685; A61B 17/00; A61B 2017/00969

(57) ABSTRACT

Disclosed are miniaturized, wearable microneedle biosensor devices, systems, and methods for measuring intradermal concentrations of various metabolites (including but not limited to lactate, pyruvate, and/or glucose) in order to directly assess the viability of living tissue (e.g., human or other mammalian soft tissue). In a variety of implementations, for example, the disclosed microneedle biosensor devices are self-contained in terms of power source, sample acquisition, measurement, and data transmission capabilities, e.g., allowing it to function autonomously once placed. In some implementations, for example, the disclosed microneedle biosensor devices can assess the viability of soft tissue (e.g., skin with or without fat, fascia, or muscle) during and after reconstructive surgery in which soft tissue is repositioned, rotated, or transferred to another site in the body (including but not limited to both microvascular free tissue transfer and regional pedicled flaps).

### TECHNICAL FIELD

This patent document relates to biosensor devices, systems, and methods, and particularly to microneedle sensors.

### BACKGROUND

Biosensors can provide real-time detection of physiological substances and processes in living things. A biosensor is an analytical tool that can detect a chemical, substance, or organism using a biologically sensitive component coupled with a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents.

### SUMMARY

Disclosed are miniaturized, wearable microneedle biosensor devices, systems, and methods for measuring intradermal concentrations of various metabolites (including but not limited to lactate, pyruvate, and/or glucose) in order to directly assess the viability of living tissue (e.g., human or other mammalian soft tissue). In a variety of implementations, for example, the disclosed microneedle biosensor devices are self-contained in terms of power source, sample acquisition, measurement, and data transmission capabilities, e.g., allowing it to function autonomously once placed. In some implementations, for example, the disclosed microneedle biosensor devices can assess the viability of soft tissue (e.g., skin with or without fat, fascia, or muscle) during and after reconstructive surgery in which soft tissue is repositioned, rotated, or transferred to another site in the body (including but not limited to both microvascular free tissue transfer and regional pedicled flaps).

In some embodiments, for example, a biosensor device in accordance with the present technology includes a microneedle array containing multiple electrochemical sensors at different tips of the microneedle array, which can produce simultaneous parallel electrical measurements of different target metabolites, e.g., for assessing the viability of transplanted tissues in real-time, before, during and after its transfer (after free flap surgery). The microneedle electrode tips can be functionalized with a selective bioreceptor for recognizing and detecting the target metabolites.

The disclosed microneedle biosensor devices can allow for (i) direct assessment of the viability of the human or mammalian soft tissue; (ii) direct assessment of the viability of transplanted tissue in real-time, before, during and after the transplant; and (iii) direct assessment of tissue health through biosensing of metabolites. The device is further advantageous in alerting the surgeons to early problems, thereby increasing the surgical salvage rate.

In some aspects, a method for assessing tissue viability during a tissue transplantation includes attaching a wearable sensor device comprising at least one microneedle having an electrochemical sensor electrode to detect an electrical signal from a reaction with a target analyte in a biofluid exposed to the at least one microneedle, wherein the wearable sensor device is attached to a tissue of an anatomic structure of a subject at an outer surface of the anatomic structure while at a first body location of the subject; measuring a first intradermal concentration of at least one metabolite from the tissue at the first body location of the subject; and detaching at least a portion of the anatomic structure that includes the tissue with the attached wearable sensor device, thereby forming a detached body part with the wearable sensor device attached to the outer surface of the detached body part; attaching the detached body part at a second body location of the subject; and measuring a second intradermal concentration of the at least one metabolite from the tissue at the second body location of the subject.

In some aspects, a method for assessing tissue viability during a tissue transplantation includes attaching a wearable sensor device comprising at least one microneedle having an electrochemical sensor electrode to detect an electrical signal from a reaction with a target analyte (or multiple analytes simultaneously) in a biofluid exposed to the at least one microneedle, wherein the wearable sensor device is attached to a tissue of an anatomic structure of a subject at an outer surface of the anatomic structure while at a first body location of the subject; measuring a first intradermal concentration of at least one metabolite from the tissue at the first body location of the subject; and detaching at least a portion of the anatomic structure that includes the tissue with the attached wearable sensor device, thereby forming a detached body part with the wearable sensor device attached to the outer surface of the detached body part; attaching the detached body part at a second body location of the subject; and measuring a second intradermal concentration of the at least one metabolite from the tissue at the second body location of the subject. For example, the target analyte or multiple analytes can include one of or some of or all of glucose, lactate, or pyruvate.

In some aspects, a method for assessing tissue viability during a tissue transplantation includes obtaining, from a wearable sensor device exposed to a biofluid in a tissue of a subject while the tissue is at a first body location of the subject, a first dataset comprising at least one intradermal concentration of one or more analytes (e.g., glucose, lactate, and/or pyruvate) from the tissue at the first body location of the subject; obtaining, from the wearable sensor device exposed to the biofluid in the tissue of the subject while the tissue is at a second body location of the subject, a second dataset comprising at least one intradermal concentration of the one or more analytes from the tissue at the second body location of the subject, wherein, prior to obtaining the second data set, the tissue was detected from the first body location and transplanted at the second body location of the subject; and comparing, by a data processing unit configured to process data obtained from the wearable sensor device, the first dataset to the second dataset, wherein the comparing includes correlating temporal information in the first dataset and the second dataset to temporal stages of the tissue transplantation, wherein the at least one intradermal concentration of the one or more analytes at the first body location of the subject and the at least one intradermal concentration of the one or more analytes at the second body location of the subject are measured by one or more microneedle electrochemical sensor electrodes of the wearable sensor device.

The subject matter described in this patent document can be implemented in specific ways that provide one or more of the following features.

### BRIEF DESCRIPTION OF THE DRAWINGS

FIG. 1 shows a block diagram of an example embodiment of a wearable microneedle biosensor device in accordance with the present technology.

FIG. 2 shows a diagram depicting an example method for assessing tissue viability during a tissue transplantation in accordance with the present technology.

FIG. 3 shows images of example tissues on a subject that was assessed for tissue viability using conventional techniques, such as a physical examination.

FIG. 4 shows an example embodiment of a wearable microneedle biosensor device in accordance with the present technology.

FIG. 5 shows a diagram depicting an example of a microneedle electrochemical biosensor in accordance with the present technology.

FIG. 6 shows images and schematic representations of example embodiments of a microneedle sensor unit of a wearable microneedle biosensor device in accordance with the present technology.

FIG. 7 shows a schematic representation of an example embodiment of a wearable microneedle biosensor device in accordance with the present technology.

FIG. 8 shows a schematic representation of an example embodiment of a wearable microneedle biosensor device for quantitative, continuous tissue viability monitoring in accordance with the present technology.

FIG. 9 shows an image of an example surgical implementation of a wearable microneedle biosensor device in accordance with the present technology.

FIG. 10 shows a flow diagram depicting an example method for assessing tissue viability during a tissue transplantation in accordance with the present technology.

### DETAILED DESCRIPTION

Free flap surgery, also called free tissue transfer, is a reconstructive surgery technique that involves the transfer of tissue (e.g., skin) from one area of the body to another. Presently, free flap reconstructive surgery can often be curative of a tumor (e.g., cancerous tumor) in the head or neck, breast, or other anatomical region, e.g., sometimes on its own or in combination with chemotherapy and/or radiation therapy. However, there are significant risks in free flap surgeries, including loss or death of the flap due to complications from the free flap tissue transfer.

For example, microvascular free tissue transfer (MFTT) surgery is an advanced reconstructive procedure performed for patients after oncologic surgery (e.g., breast reconstruction after mastectomy), trauma, or other conditions in which vascularized soft tissue coverage is needed to obliterate a dead space or cover bone. For the first week after surgery, the flap is at risk of failing from venous or arterial compromise. Urgent surgical intervention may salvage the flap tissue, but the likelihood of successful salvage decreases when problems are detected late. When a flap totally fails, the result is devastating and has significant financial consequences, including need of a second reconstruction procedure, increased length of hospitalization, and patient morbidity. Thus, early identification of flap failure is an essential prerequisite for flap salvage. The current standard of care typically necessitates a one to two day ICU stay where nurses check audible doppler and physical exams hourly, and ICU stays are extremely expensive. These metrics are qualitative and the sensitivity for detecting early compromise is low.

Thus, it would be beneficial to provide a device, technique, and/or system that can continuously monitor in real time the viability of tissue before, during, and after transport.

Furthermore, the need for continuous, minimally invasive measurements of local and systemic analytes is essential in translational medicine. Assessment of continuous whole system or tissue-specific analytes in large patient populations is a clinical and translational science roadblock for the monitoring and treatment of complex disease. As an example, tissue viability is an important factor in the assessment and care of patients with a wide range of disorders, including peripheral vascular disease, trauma, critical limb ischemia, and breast, skin, or head and neck cancers requiring reconstruction with MFTT. Although critical to the decision and timing of surgical or pharmacologic intervention, rapid and accurate assessment of the end-tissue's microcirculatory arterial perfusion and venous drainage is a challenging task, which is presently limited by subjective bedside examination or imperfect surrogate tests of upstream perfusion, such as doppler ultrasound. As current methods to assess tissue viability are time-consuming, intermittent, and subjective, this leads to delayed and inaccurate translation of therapies and interventions. A continuous and quantitative sensing method to directly interrogate end-tissue viability is critically needed for both clinical care and clinical translational research.

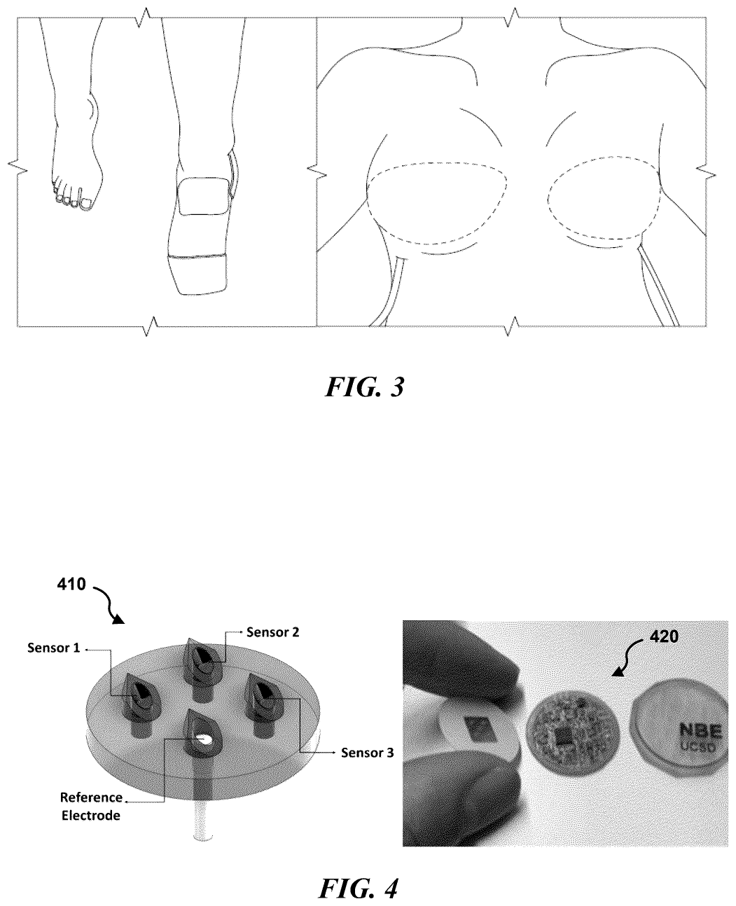

Rapid and accurate assessment of tissue viability is critical for clinicians to determine the type and timing of intervention to prevent morbidity and mortality. For example, critical limb ischemia resulting from unstable peripheral vascular disease or trauma may benefit from surgical intervention, both as a preventative measure and to identify and best salvage tissue during amputation (see FIG. 3, left image). The procedure, timing, and location of intervention depends on the vital status of that limb. Similarly, in compartment syndrome, elevated pressure can limit blood flow to muscles and nerves. As another example, following a MFTT reconstruction following mastectomy, other oncologic surgery, trauma, burn, or other wound, a free flap's soft tissue skin paddle might show subtle and questionable signs of venous congestion, but whether to initiate pharmacologic treatment or a return to the operating room to re-explore the anastomosis crucially depends on the timing and extent of the problem. More broadly, tissue viability assessment could benefit peripheral vascular disease in general through continuous early detection and more informed intervention.

Currently, objective, quantitative, and direct methods of tissue interrogation for viability assessment in MFTT are lacking, and this gap impacts both clinical care and scientific investigation. The clinical standard-of-care is bedside assessment by trained providers with physical examination. Specifically, the relevant components of this physical exam comprise assessment of the tissue's color, temperature, turgor, and capillary refill. Tissues that are perfectly healthy and tissues that have obvious venous congestion will have clearly different characteristic appearances that even a lay-person could distinguish. For example, when assessing the transferred tissue after a breast reconstruction, advanced venous congestion can be readily identifiable for some clinical cases (see FIG. 3, right image). However, the challenge in most clinical contexts is that most cases fall into a gray zone somewhere between perfectly healthy and completely necrotic, and, importantly, physical exam findings can lag underlying physiologic changes leading to a delay in diagnosis. Additionally, the subjectivity of the assessment is affected by the provider's experience level (noting that even skilled providers with high level of experience can find making an accurate assessment to be challenging).

FIG. 3 shows images of example tissues on a subject that were assessed for tissue viability by conventional means, i.e., physical examination by a clinician. Notably, assessing the tissue viability of tissue to be transferred on a patient can be greatly improved by using a wearable microneedle biosensor device in accordance with the present technology, discussed in detail below.

Subjective assessment of physical examination components is further impacted by real-world variables that can confound accurate assessment of each component (Table 1). Tissue color can be confounded by ambient or examination lighting and racial pigmentation differences that limit the ability to recognize subtle hue changes caused by early arterial insufficiency or venous congestion. For example, the subtle whitish hue of arterial ischemia and subtle purple hue of early venous ischemia is more challenging to identify in patients with darker skin. Also, visualization and color assessment of internal (e.g., intraoral) flaps is challenged by variable and inconsistent illumination as medical providers use hand-held flashlights and patients typically have difficulty opening their mouths wide in the post-operative setting. Tissue turgor and capillary refill assessment can be confounded by the rapid swelling and fluid shifts that occur following local tissue trauma, whether it be iatrogenic (surgery) or from an external trauma, and comorbid cardiac, renal, and vascular conditions that cause fluid third-spacing or edema from venous insufficiency. Tissue temperature is a late sign as efforts by medical personnel are typically directed at artificially maintaining the patient's temperature throughout with blankets and other warming efforts. Also, the defect site to be reconstructed impacts the extent to which a temperature change would occur with ischemia-internal (e.g., intraoral) flaps are in a heated and humidified environ and thus will cool more slowly than would cutaneous (e.g., breast) flaps. Finally, all of these components are also confounded by systemic processes that can be co-occurring, such as systemic hypovolemia or anemia. For all of these reasons, a more objective and quantitative method of tissue viability assessment is desired.

TABLE 1
Confounding variables during subjective
physical examination assessment.

| Subjective Assessment | Confounding Variable |
|---|---|
| Tissue color | Lighting, race |
| Capillary refill | Lighting, race |
| Tissue turgor | Swelling, trauma |
| Tissue temperature | Environment (e.g., intraoral, cutaneous) |

Other existing techniques that have been introduced to supplement physical examination include point-of-care acoustic doppler ultrasound, implantable doppler systems, and vascular perfusion studies. Point-of-care acoustic doppler ultrasound is a widely available clinical tool that can supplement physical examination and partly addresses the aforementioned problems of objectivity and time delay between physiologic change and exam findings in the end-organ (soft tissue skin paddle). Acoustic doppler ultrasound uses ultrasonic soundwaves to detect the presence of blood flow through arteries, veins, and even distal, smaller perforating vessels, and then produces an audible sound that correlates with the amount of movement it detects. When blood flow can no longer be detected through a vessel that previously demonstrated flow, the examiner is alerted to possible critical ischemia that can or has compromised tissue viability.

Although an important part of standard assessment, acoustic doppler ultrasound suffers from two main limitations. First, while less subjective than physical examination, the technique is still intermittent, operator dependent and only semi-objective. Similar to physical examination, a delay in diagnosis can occur between examinations, and the test is only semi-objective because coarse changes are readily detectable (for example, lack of flow in the superficial femoral artery), but subtle findings are sensitive to the examiner's experience level (smaller perforators are exquisitely sensitive to the location, angle, and pressure of the handheld probe, and the acoustic output may change in character but not volume for early changes). Second, and more importantly, it makes physiologic assumptions in evaluating only a surrogate for tissue viability, rather than direct tissue assessment. The main assumption is that the tissue's viability is largely dependent on that discrete vessel, which can be influenced by geographic miss (the vessel moved after changes to patient positioning or the examiner is actually detecting flow in a larger vessel deep to the expected target location) and by the non-exclusive nature of vascular supply (watershed areas between perforator territories). Furthermore, this surrogate measurement cannot account for upstream changes at the level of the blood vessel that have not (or perhaps will not) propagate downstream to the end organ (skin paddle), such as intermittent vasospasm.

More recently, products, such as implantable doppler systems, that attempt to address the limitations of intermittent examination and geographic miss have reached the market. However, the use of an implantable doppler is only possible for tissue viability following MFTT, as the implantable doppler probe must be placed around the vessel at the time of surgery. Furthermore, implantable doppler systems still suffer from false positives (if the sensor loses direct contact with the vessel wall it will not detect blood flow) and false (or delayed) negatives (continued arterial inflow despite insufficient venous outflow, or arterial insufficiency at the level of downstream perforating vessels).

Another state-of-the-art technique involves systems comprising a systemically injectable near-infrared fluorescent dye (such as indocyanine green) and fluorescent cameras to visualize blood flow within tissues. Although such perfusion studies are an excellent adjunct for many intraoperative cases, the resource-intensive (cost, personnel, equipment, space) nature of this procedure limits its widespread adoption into clinical practice or the research environment outside of the operating theater. Additionally, all three of these techniques to assess blood flow only measure an upstream surrogate of tissue viability and cannot directly report the severity of tissue compromise or health of the tissue itself.

These examples above on the limitations of physical examination and existing adjunct modalities (point-of-care ultrasound, implantable ultrasound, and vascular perfusion studies) discuss the impact on clinical care, but scientific investigation closely follows, as it uses the same instruments to evaluate a treatment course's impact.

Microdialysis is one technique that allows direct metabolic interrogation of interstitial tissue for the assessment of that tissue's viability. In the context of MFTT, microdialysis is a system that involves the surgical insertion of a double lumen catheter with a semi-permeable dialysis membrane into the subcutaneous adipose tissue of the flap. It is then connected to an infusion pump which collects, over a 15- to 20-minute period, dialysate that reflects the composition of the interstitial fluid. Over the past 2-3 decades, tissue metabolism as monitored by microdialysis has shown some promise for monitoring after MFTT. For example, it has been found that glucose, pyruvate, and lactate may reliably predict not only the presence of tissue ischemia, but may distinguish venous from arterial causes. Also, microdialysis has been shown to detect metabolic changes predictive of ischemia earlier than standard examination, giving surgeons critical lead time that can increase the chance of a successful surgical intervention for flap salvage. Furthermore, the accuracy in terms of sensitivity and specificity is superior to clinical examination, external doppler ultrasound, and implantable doppler sensors.

Despite technique feasibility and demonstrated improvement over other conventional techniques, microdialysis techniques have not garnered mainstream clinical adoption. This is because microdialysis techniques are critically limited in several ways. First, microdialysis is time consuming and resource-intensive and requires frequent dialysate collection and interpretation from trained medical personnel. Second, it is an invasive technique that carries risk of complications for a healing flap, including bleeding and hematoma, and requires specific training on a procedure that is unfamiliar to most surgeons. Third, by nature it provides only a single time-point of data that, even if repeated, is intermittent and there is a considerable amount of time that elapses while the fluid is collected, analyzed by an external machine, and data uploaded to the requesting entity. Fourth, it logistically complicates post-operative nursing care for what is already a challenging and specialized patient by adding additional devices, lines, and wires on the patient's surgical site—for example, following MFTT, it is typical for the patient to have 1-4 surgical drains at the recipient site, 1-2 surgical drains at the donor site, 2 intravenous catheters, an arterial catheter, a urinary catheter, and sometimes a tracheostomy tube and nasogastric feeding tube.

Thus, a device, technique, and system that can continuously monitor in real time the viability of tissue before, during, and after transport will be transformative for tissue transfer surgery and for national translational research pipelines.

Disclosed are wearable microneedle biosensor devices, systems, and methods for measuring intradermal concentrations of various metabolites (including but not limited to lactate, pyruvate, and/or glucose) in order to directly assess the viability of living tissue (e.g., human or other mammalian soft tissue). In a variety of implementations, for example, the disclosed microneedle biosensor devices are self-contained in terms of power source, sample acquisition, measurement, and data transmission capabilities, e.g., allowing it to function autonomously once placed. In some implementations, for example, the disclosed microneedle biosensor devices can assess the viability of soft tissue (e.g., skin with or without fat, fascia, or muscle) during and after reconstructive surgery in which soft tissue is repositioned, rotated, or transferred to another site in the body (including but not limited to both microvascular free tissue transfer and regional pedicled flaps).

In some embodiments, for example, a biosensor device in accordance with the present technology includes a microneedle array containing multiple electrochemical sensors at different tips of the microneedle array, which can produce simultaneous parallel electrical measurements of different target metabolites, e.g., for assessing the viability of transplanted tissues in real-time, before, during and after its transfer (after free flap surgery). The microneedle electrode tips can be functionalized with a selective bioreceptor for recognizing and detecting the target metabolites. For various embodiments, for example, the microneedle(s) of the disclosed biosensor device, e.g., a microneedle sensor array, can be fabricated using different fabrication techniques and can be configured with different numbers of microneedle sensors of different geometries, dimensions, spacings and layouts.

The disclosed wearable microneedle biosensor devices, systems, and methods have the promise to alert surgeons to early problems with their free flaps to increase the surgical salvage rate, and also has the potential to shorten or eliminate ICU stays.

For example, the unique combination of metabolic analytes allows the disclosed wearable microneedle biosensor device to directly assess the viability of the tissues, rather than use a surrogate variable, such as blood flow, to extrapolate tissue health. Furthermore, the small size and wireless and autonomous capabilities can allow the disclosed wearable microneedle biosensor device to be used during MFTT surgery both intraoperatively and post-operatively in order for surgeons and other healthcare providers to monitor the free flap during and after surgery.

Notably, the standard of care for tissue viability assessment is physical examination. This is sometimes supplemented by tools that allow measurement of blood flow as a surrogate variable to extrapolate tissue health (e.g., ultrasound doppler with handheld probe, ultrasound doppler with implantable probe, fluorescent dye vascular profusion studies). None of these techniques directly assess tissue health through biosensing of its metabolites.

Moreover, while microdialysis techniques may be a conventional means to assess tissue health, microdialysis is a manual technique in which a healthcare provider aspirates fluid from the intradermal or subcutaneous spaces and then transports the specimen to a separate lab where machines will measure the concentrations with traditional techniques. Microdialysis and other conventional techniques lack autonomous devices that can assess tissue viability in real time, continuously, and without the intervention of clinicians.

The embodiments described herein overcome the limitations of traditional intermittent microdialysis by providing a wireless, wearable microneedle intradermal sensing platform for real-time, continuous and autonomous tracking of multiple metabolic parameters to quantitatively assess end-tissue viability, and a platform to scale this technology to other use cases. In some implementations, MFTT is used as the ideal model system use case because timing of the ischemic insult (arterial and venous ligation) is precisely known, complete, and there is a perfectly matched internal control (the contralateral donor site of the same patient).

The design and fabrication of a wearable, self-powered, multiplexed microneedle sensor device capable of electrochemical detection of multiple metabolites in free tissue transfer surgery is described herein. In one example application, the microneedle sensor device can be used for clinical detection of tissue metabolite levels intraoperatively during routine MFTT and during the post-operative period following such surgery. In some cases, the microneedle sensor device can predict tissue ischemia as soon as or earlier than detection by standard-of-care clinical methods (physical examination and external doppler ultrasound).

The disclosed wearable microneedle biosensor devices, systems, and methods can be implemented for a variety of applications because critical limb ischemia or soft tissue compromise may occur following a traumatic injury and this can help the treating providers with their assessment of tissue viability.

Furthermore, the disclosed wearable microneedle biosensor devices, systems, and methods can be implemented for a variety of applications for the scientific community because the disclosed technology can empower researchers with the ability to test more nuanced hypotheses than “is the tissue alive or dead, blood flow or no blood flow,” e.g., by giving a higher resolution outcome into tissue health that may help accelerate the development of new pharmacologic interventions or treatment paradigms.

The disclosed wearable microneedle biosensor devices, systems, and methods have important implications not only for the evaluation and treatment after MFTT, but also for accelerating clinical and translational science. Alongside certain example specific use cases described in this disclosure, the disclosed wearable transplantable tissue viability sensor technology will be applicable to tissue viability monitoring for other indications including peripheral vascular disease, trauma, and reconstruction following burns. In some implementations, the example embodiments in accordance with the disclosed technology are capable of simultaneously and continuously detecting dozens of unique analytes and are thus broadly generalizable to clinical indications including, but not limited to, diabetes, Parkinson's disease, sepsis and shock, coronary artery disease, kidney disorders, antibiotic and other pharmacologic monitoring. In some implementations, the disclosed wearable microneedle biosensor devices, systems, and methods will be able to accelerate its clinical use for both translational research and patient care.

The disclosed embodiments aim to address the clinical and translational science barrier of continuous and objective/reliable analyses of system-/tissue-specific analytes. In an example use case to be discussed in further detail, a wireless, wearable intradermal microneedle sensor array for real-time, continuous tracking of multiple metabolic parameters is disclosed and employed to assess tissue viability. The rapid and accurate assessment of tissue arterial perfusion and venous drainage has broad applicability to peripheral vascular disease, breast cancer, skin cancer, and head and neck cancers, and to the fields of vascular surgery, plastic reconstructive surgery, and head and neck oncologic and reconstructive surgery. MFTT is proposed as the ideal model system use case because timing of the ischemic insult (arterial and venous ligation) is precisely known, complete (e.g., 0 to 100% occlusion), and there is a perfectly matched internal control (the contralateral donor site of the same patient).

Among other features and benefits, the disclosed wearable microneedle biosensor devices, systems, and methods have the potential to both improve these disease conditions and accelerate clinical and translational science. For example, devices, systems, and methods would have immediate clinical impact on the field in terms of improved outcomes, safety, and cost-effectiveness. Second, the devices, systems, and methods would empower investigators with a titratable and more nuanced signal to serve as a primary or secondary outcome in their clinical trials. And third, the devices, systems, and methods represents a platform technology—not only is METT a use case for the specific tissue viability sensor, the chosen metabolites (e.g., lactate, pyruvate, and glucose) are a use-case for a more generalizable approach to multiplexed microneedle intradermal sensing as a more rapid, tele-capable, and minimally-invasive method to approximate systemic metabolite levels than venipuncture, which might allow access to previously unreachable patient populations, or trial designs that leverage the remote and autonomous lab-on-a-chip nature of data acquisition. Moreover, the disclosed embodiments hold promise for monitoring various diseases, accelerating translational research, and enhancing patient care. Thus, the disclosed embodiments are vital to the development, implementation, scaling, and dissemination of this biosensor technology to address urgent public health needs and translate science into standard of care.

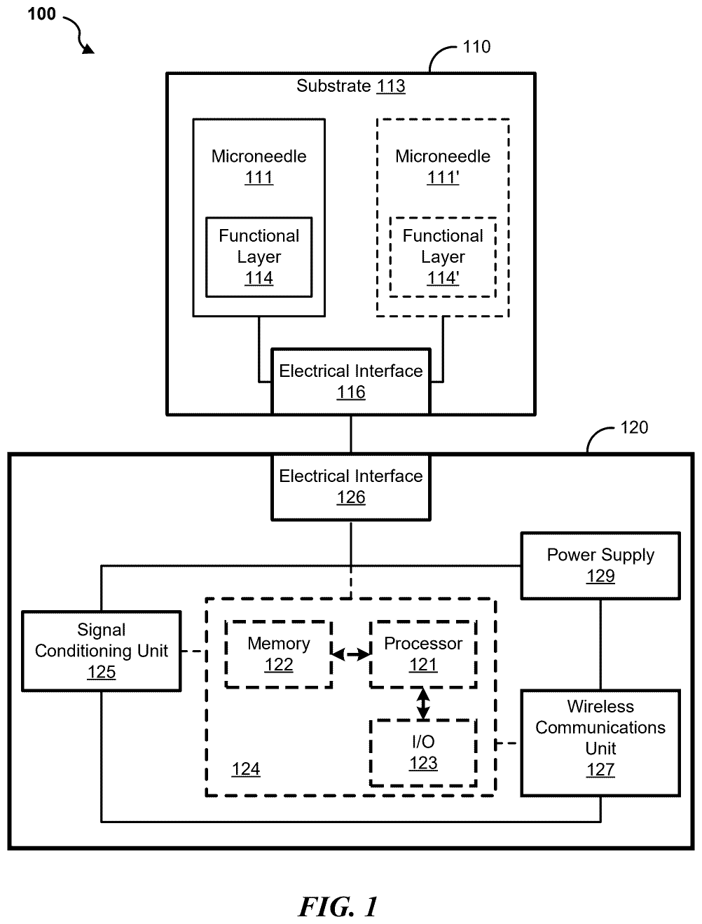

FIG. 1 shows a block diagram of an example embodiment of a wearable microneedle biosensor device 100 in accordance with the present technology. The wearable microneedle biosensor device 100 includes a sensor unit 110 that includes a substrate 113, at least one microneedle 111 that is arranged on the substrate 113. In some embodiments of the device 100, the wearable microneedle biosensor device 100 includes two or more microneedles (i.e., shown as microneedle 111 and microneedle 111′), which the two or more microneedles can be configured in an array of microneedles. The at least one microneedle 111 includes a base structure and a tip region at an outward end of the microneedle 111 with respect to the substrate 113. The at least one microneedle 111 is configured to include an electrochemical sensor electrode disposed at the tip region, e.g., to detect an electrical signal from a reaction with a target analyte in a biofluid exposed to the microneedle 111. The sensor unit 110 includes an electrical interface 116 that is electrically connected to the at least one electrochemical sensor electrode of the microneedle 111 (and electrochemical sensor electrode(s) of other (optional) microneedle(s) 111′). In such embodiments, for example, the electrochemical sensor electrode may be functionalized by a chemical layer (functional layer 114) to interact with the target analyte in the biofluid in order to produce an electrical signal associated with the reaction that is detectable at the electrochemical sensor electrode. For example, in some embodiments, the functional layer 114 can be deposited on just a portion of a microneedle 111, e.g., such as the tip region (e.g., the apex of the tip and/or below the apex of the tip); and/or, in some embodiments, the functional layer 114 can be coated on the outer wall of the body of the microneedle 111. In various embodiments, the functional layer 114 is configured to chemically facilitate an electrochemically detectable reaction with a target analyte.

In various implementations, for example, the target analyte can include a chemical substance that is associated with a biomarker. For example, the target analyte can include a metabolite, electrolyte, protein, amino acid, nucleic acid, lipid, liposome, nanoparticle, and/or therapeutic drug. In some examples of a metabolite, the target analyte can include a ketone body. In some examples of a protein target analyte, the target analyte can include an enzyme, peptide-based aptamer, antibody, or hormone. In some examples of a nucleic acid target analyte, the target analyte can include a nucleotide, oligonucleotide, oligonucleotide-based aptamer, deoxyribonucleic acid (DNA) or portion thereof, and/or ribonucleic acid (RNA) or portion thereof. In various implementations, for example, the biofluid containing the target analyte can include interstitial fluid, transdermal fluid, intraocular fluid, vitreous humor, cerebrospinal fluid, extracellular fluid, plasma, serum, lacrimal fluid, saliva, perspiration, mucus, and/or blood.

The wearable microneedle biosensor device 100 includes an electronics unit 120 that includes a signal conditioning unit 125, a power supply 129, an output unit (e.g., which can be embodied as a wireless communications unit 127), and an electrical interface 126 (e.g., which can include one or more electrical interconnections, such as pins). The electronics unit 120 is configured to receive (e.g., at the electrical interface 126) and at least partially process electrical signals acquired from the sensor unit 110 (e.g., at the signal conditioning unit 125). In some embodiments, for example, the electrical interface 126 is configured to electrically couple to output ports of the electrical interface 116 of the sensor unit 110. The electronics unit 120 is configured to output the raw or partially processed electrical signals and/or processed data. In some embodiments, the electronics unit 120 of the device 100 may include an optional data processing unit 124 to process the at least partially processed signals as data, e.g., in digital format. For example, in some implementations, the data processing unit 124 can include a microcontroller and multiplexer to manage data acquisition on data channels from the electrodes.

In some embodiments of the electronics unit 120, for example, the signal conditioning unit 125 can include an electrical circuit including one or more amplifier(s) and filter(s) to condition the raw detected electrical signals from the electrode(s) of the microneedle(s) 111 of the sensor unit 110, e.g., improving signal-to-noise ratio. In some implementations, the signal conditioning unit 125 can include drive circuitry for operating an electrochemical sensing technique performed at the electrode(s) of the microneedle(s) 111 of the sensor unit 110 to implement the desired sensing mode for detecting the analyte from the biofluid.

In some embodiments, for example, the electronics unit 120 can include electrical contacts that electrically interface with an electrical conduit to provide the data to an external circuit or device. In some embodiments, for example, the output unit can include a wireless communications unit 127 that includes a wireless transmitter or transceiver device, e.g., capable of communicating with an external device to provide raw, partially-processed, or fully-processed data from the data processing unit 124. An example transceiver unit can include a BLE chipset to communicate with a BLE-enabled device, such as a smartphone. The power supply 129 can include a battery, fuel cell or other power source to supply power to the components of the electronics unit 120 and/or the sensor unit 110.

In some embodiments of the electronics unit 120, for example, the (optional) data processing unit 124 can include a processor 121 to process data, and memory 122 in communication with the processor 121 to store and/or buffer data. For example, the processor 121 can include a central processing unit (CPU) or a microcontroller unit (MCU). For example, the memory 122 can include and store processor-executable code, which when executed by the processor, configures the data processing unit 124 to perform various operations, e.g., such as receiving information, commands, and/or data, processing information and data, and transmitting or providing information/data to another device. To support various functions of the data processing unit 124, the memory 122 can store information and data, such as instructions, software, values, images, and other data processed or referenced by the processor 121. For example, various types of Random Access Memory (RAM) devices, Read Only Memory (ROM) devices, Flash Memory devices, and other suitable storage media can be used to implement storage functions of the memory 122. In some implementations, the data processing unit 124 includes an input/output (I/O) unit 123 to interface the processor 121 and/or memory 122 to other modules, units or devices, e.g., associated with the mobile device 130, the remote data processing system 140, and/or other external devices. In some embodiments, the processor 121, memory 122, and/or I/O unit 123 is in communication with the wireless communications unit 127, e.g., such as a transmitter (Tx) or a transmitter/receiver (Tx/Rx) unit. For example, in such embodiments, the I/O unit 123 can interface the processor 121 and memory 122 with the wireless communications unit 127, e.g., to utilize various types of wireless interfaces compatible with typical data communication standards, which can be used in communications of the data processing unit 124 with other devices, e.g., such as between the one or more computers in the cloud and the user device. The data communication standards include, but are not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G/6G cellular communication methods, and parallel interfaces. In some implementations, the data processing unit 124 can interface with other devices using a wired connection via the I/O unit 123. The data processing unit 120 can also interface with other external interfaces, sources of data storage, and/or visual or audio display devices, etc. to retrieve and transfer data and information that can be processed by the processor 121, stored in the memory 122, or exhibited on an output unit of the mobile device 130 (e.g., smartphone) or an external device.

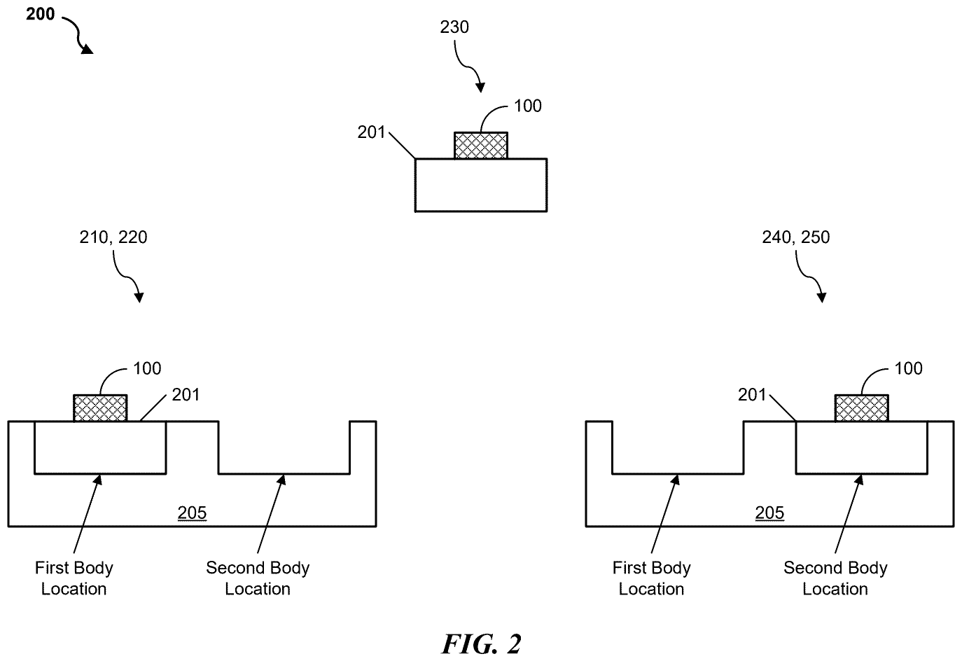

FIG. 2 shows a diagram depicting an example method 200 for assessing tissue viability during a tissue transplantation in accordance with the present technology. The method 200 includes a process 210 to attach an example embodiment of the wearable sensor device 100 to a tissue 201 of an anatomic structure 205 of a subject at an outer surface of the anatomic structure while at a first body location of the subject. The method 200 includes a process 220 to measure a first intradermal concentration of at least one metabolite from the tissue 201 at the first body location of the subject. The method 200 includes a process 230 to detach at least a portion of the anatomic structure 205 that includes the tissue with the attached wearable sensor device 100, thereby forming a detached body part with the wearable sensor device 100 attached to the outer surface of the detached body part. The method 200 includes a process 240 to attach the detached body part at a second body location of the subject. The method 200 includes a process 250 to measure a second intradermal concentration of the at least one metabolite from the tissue 201 at the second body location of the subject.

In some embodiments of the method 200, measurements of the first and the second intradermal concentrations are measured continuously during when the tissue 201 (e.g., at least a portion of the anatomic structure) is at the first body location and during when the tissue 201 (e.g., at least a portion of the anatomic structure) is attached to the second body location. In some embodiments of the method 200, the method 200 includes a process to measure a third intradermal concentration of the at least one metabolite from the tissue 100 during the process 230, e.g., between the detaching the at least a portion of the anatomic structure from the first body location of the subject and the attaching the detached body part at the second body location of the subject. For example, in such embodiments, the measurements of the first, the second, and the third intradermal concentrations are measured continuously during the detaching of the at least a portion of the anatomic structure from the first body location of the subject and the attaching of the detached body part at the second body location of the subject.

FIG. 4 shows example embodiments of the sensor unit 110 and the electronics unit 120 for the wearable microneedle biosensor device 100 introduced in FIG. 1, labeled sensor unit 410 and electronics unit 420, respectively. In some implementations, the wearable microneedle biosensor device is powered by one or more biofuel cells (not shown) and implemented for the simultaneous electrochemical (amperometric) detection of various metabolites (e.g., lactate, glucose, and pyruvate) present within skin interstitial fluid. As shown in FIG. 4 (left diagram), the sensor unit 410, in one implementation, comprises multiple microneedle sensors each associated with a specific metabolite (e.g., lactate, glucose, or pyruvate, corresponding to Sensor 2, Sensor 3, and Sensor 1, respectively), where the microneedle sensors each include a microneedle and an electrode. In some implementations, the electrode can comprise Ag and Cl, e.g., such as an Ag/AgCl electrode for electrochemical sensing applications. The electronics unit 420 may include a wireless circuit board and a circuit board covering to cover the wireless circuit board as shown in FIG. 4 (right, image). In some implementations, the electronics unit 420 includes a transceiver configured to transmit electrical signals from the electrodes of the microneedle sensors to a device (e.g., a computer) for analysis or display.

In some implementations of the wearable microneedle biosensor device 100, the microneedle design parameters and resulting dimensions and layout are optimized to ensure robust operation. For example, the wearable microneedle biosensor device 100 can be optimized for continuous monitoring lasting 4-5 days, which is the typical duration of free flap monitoring following microvascular free tissue transfer surgery. Because it can be applied to patients intraoperatively, the device is also able to tolerate surgical sterilization.

The microneedle sensors can be fabricated using well-defined micro-injection molding or lithographic techniques using biocompatible polymeric or silicon materials and can be integrated with wireless electronics on an epidermally wearable platform. The wearable microneedle biosensor device 100 can be thoroughly sterilized prior to use according to clinical use protocols and secured to the surgical site of a subject with the entire system including non-toxic, FDA-approved materials. Additionally, the microneedle sensors can be functionalized with biocompatible enzymatic and polymeric reagent layers to ensure reliable, stable and selective operation.

In some implementations, the wearable microneedle biosensor device 100 is used for real-time, continuous transplantable tissue viability monitoring and configured to include hollow microneedle sensors, each including a microneedle structure and an electrode (e.g., which may be chemically modified for target analyte detection). Upon fabrication of the hollow microneedles, wires can be inserted into the microneedle lumen through press-fitting for electronics attachment and to provide an electrode surface available for subsequent functionalization though the microneedle aperture. Alternatively, for example, the microneedle lumen may be filled with a conductive, biocompatible carbon paste as the transduction element. For micro-injection molded microneedles, a medical-grade liquid crystal polymer can be used to ensure suitable mechanical characteristics. Alternatively, micromolding can be used for fabricating conically-shaped solid polymeric microneedles, which can be sputtered with Pt or Au films to create electrode-transducer tips on which the reagent enzyme layer will be immobilized. Microneedle shape, dimensions, and spacing can be critically evaluated to ensure optimal performance of continuous assessment of interstitial fluid analytes. It is noted that the wearable microneedle biosensor device 100 for real-time, continuous transplantable tissue viability monitoring can be configured to include solid microneedle sensors in addition to or alternatively from the hollow microneedle sensors.

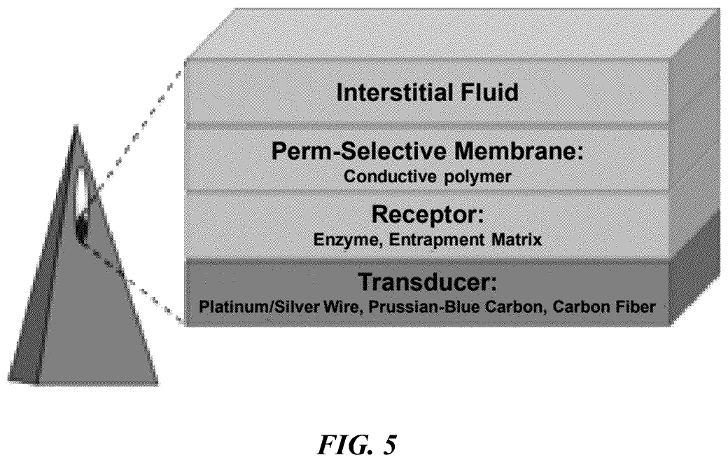

FIG. 5 shows a diagram depicting an example of a microneedle sensor for example embodiments of the real-time, continuous-monitoring, wearable microneedle biosensor device based on the disclosed technology. As shown therein, the microneedle sensor may be an electrochemical biosensor functionalized using several coating layers, each with a specific purpose. The microneedle sensor includes a press-fitted wire transducer (e.g., platinum or silver wire, carbon fiber, etc.) that can be modified with certain additives (e.g., carbon microfibers or nanoparticles) for augmenting sensor response. In some implementations, a metallic transducer layer film and an enzyme receptor layer are separated from the interstitial fluid by a biocompatible perm-selective protective layers. In some implementations, the microneedle sensor can be configured to include three separate microneedle-based electrodes, including a silver-wire based reference electrode and working and counter electrodes with platinum or carbon fiber wires. In some implementations, the electrode transducer may be coated with an enzymatic layer and/or a perm-selective layer (e.g., a permeability-tunable conducting polymer material to control the porosity of the conductive polymer, such as, but not limited to, poly(o-phenylenediamine) (PPD)).

In some implementations of wearable, microneedle sensing devices based on the disclosed technology, continuous parallel biosensing of glucose, lactate and pyruvate, relies on immobilization of the corresponding oxidase enzymes and monitoring the current signal of the corresponding hydrogen-peroxide enzymatic reaction product. In some implementations, a bioreceptor layer (e.g., receptor layer in FIG. 5) can also incorporate an entrapment matrix (e.g., chitosan, poly-L-lysine, etc.) used to retain or stabilize the recognition element at the transducer surface. Following the enzyme immobilization, the sensor can be coated with an external, biocompatible polymer (e.g., chitosan, poly-L-lysine Nafion, etc.) or electropolymerized polymer (e.g., orthophenylenediamine (oPD)) contacting the skin to avoid leaching of recognition component and additives and protect the surface against biofouling and redox interferences. For self-powered sensing, a two-electrode configuration can be used with both anodic and cathodic microneedle electrodes enzyme/polymer-modified to generate low levels of power in response to metabolite concentration.

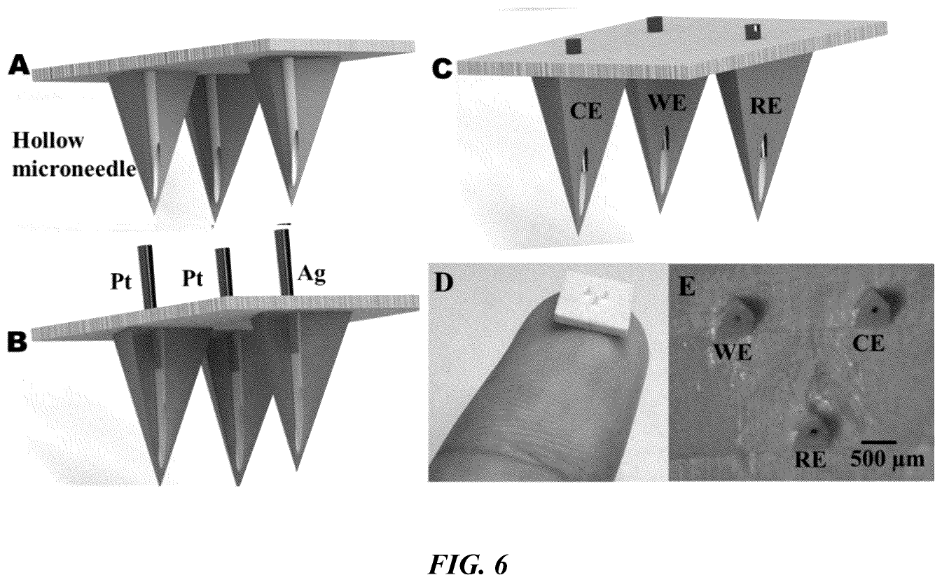

FIG. 6 shows example schematic representations of portions of an example microneedle sensor unit, in accordance with the present technology. The example microneedle sensor unit includes an example set of hollow microneedles coupled to a substrate (panel A), a set of electrode wires press-fit into a respective microneedle aperture of the set of the hollow microneedles (panel B), which include contact sites or wires from the set of electrodes to form an addressable electrode array (panel C). FIG. 6 also shows a photograph of the example microneedle sensor unit configured from the example microneedle electrode array that is attached on a user's fingertip (panel D), and an optical micrograph of the microneedle electrode array with the integrated electrode wires (panel E). In some implementations, the integrated microneedle electrode array, as shown in panel C of FIG. 6, includes a counter electrode (CE), a working electrode (WE), and a reference electrode (RE) configured to detect target metabolites. FIG. 6 panel A shows the hollow microneedles for each of the electrodes in the array. FIG. 6 panel B and panel C show Pt and Ag conductive wires that are wire press-fit into a microneedle aperture of the hollow microneedle to form the microneedle electrodes with electrically-addressable contacts. In some example embodiments, the integrated electrode array includes a first electrode wire comprising Pt which is press-fit into the CE, a second electrode wire comprising Pt which is press-fit into the WE, and a third electrode wire comprising Ag and/or Ag/Cl which is press-fit into the RE.

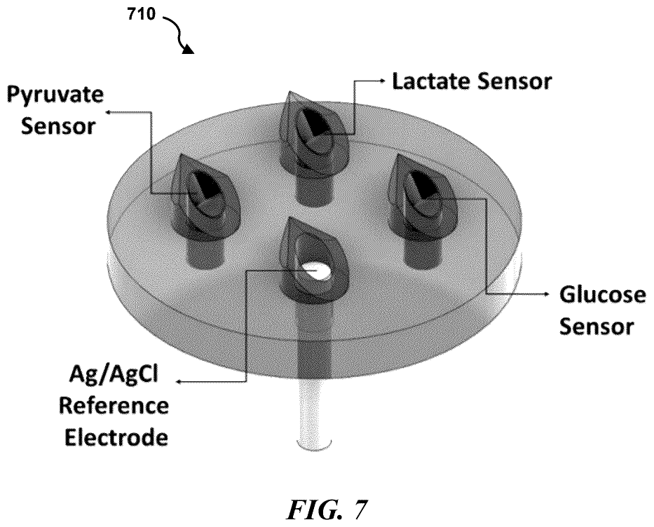

FIG. 7 shows a diagram of an example embodiment of sensor unit 110 (labeled microneedle sensor unit 710) for a wearable microneedle biosensor device in accordance with the present technology for real-time, continuous monitoring of tissue viability, particularly for transplantable tissue. The wearable microneedle biosensor device can be designed to include multiple sensors and a reference electrode. In this embodiment, for example, the microneedle sensor unit includes a first microneedle sensor electrode configured to detect pyruvate (e.g., “pyruvate sensor”), a second microneedle sensor electrode configured to detect lactate (e.g., “lactate sensor”), a third microneedle sensor electrode configured to detect glucose (e.g., “glucose sensor”), and a fourth microneedle sensor electrode configured as a reference electrode (e.g., “Ag/AgCl reference electrode), which collectively can be configured to detect tissue viability by deployment of the microneedle sensor unit on an outer skin surface of the tissue, e.g., tissue to be/that was transplanted in a subject. In some implementations, the wearable microneedle biosensor device is powered by biofuel cells developed for the simultaneous electrochemical (amperometric) detection of various metabolites (e.g., including, but not limited to pyruvate, lactate, and glucose) present within skin interstitial fluid. A schematic representation of the microneedle sensor unit for the wearable, microneedle sensing array device for simultaneous detection of tissue analyte is shown in FIG. 7. In some embodiments, the wearable microneedle biosensor device includes a microneedle component, a sensing electronic circuit breadboard, and an external casing that are fully integrated into the skin-worn device (not shown in FIG. 7). In some example embodiments, the microneedle component can include a plurality of microneedle sensor units that are distributable on the transplant tissue to continuously and in real time assess viability of various regions of the tissue individually, e.g., thereby providing a spatial-temporal mapping of the viability of the transplant tissue.

In some implementations, the wearable microneedle biosensor device is integrated with wireless electronics (e.g., the electronics unit 120). In some applications, for example, low levels of generated power will be monitored with the integrated wireless electronics to yield correlated analyte levels, without necessitating external power sources. In one example use case, the data generated by the wearable microneedle biosensor device can provide a unique assessment of a tissue's metabolic and physiological status. In some implementations, all fabrication techniques and utilized materials for the wearable microneedle biosensor device, including covering circuit board, are thoroughly evaluated for biocompatibility under the applications described herein for direct contact with the epidermis and subcutaneous interstitial fluid. In some implementations of the wearable microneedle biosensor device, interference effects derived from non-target compounds present within the interstitial fluid can be assessed. Microneedle design parameters and resulting dimensions and layout may be optimized for each application of the disclosed device to ensure robust operation for continuous monitoring. In one example application, depending on the target metabolites, the receptor layer applied to the working electrode will include specific biorecognition components such as enzymes (for detection of metabolites) or ionophores (for detection of metabolites) that have been used for several in vivo applications in humans.

Other examples of microneedle sensor devices that may be implemented in accordance with the present technology are described in U.S. Patent Publication No. 2014/0336487A1 (“Microneedle Arrays for Biosensing and Drug Delivery”), U.S. Patent Publication No. 2023/0111253A1 (“Microneedle Array Sensor Patch for Continuous Multi-Analyte Detection”), and U.S. Patent Publication No. 2023/0012662A1 (“Wearable, Non-Intrusive Microneedle Sensor”), which are incorporated by reference as part of the disclosure of this patent document for all purposes. Some examples of biofuel cell devices that can be integrated with the disclosed microneedle sensor devices in accordance with the present technology are described in U.S. Patent Publication No. 2014/0322617A1 (“Printed Biofuel Cells”), which is incorporated by reference as part of the disclosure of this patent document for all purposes.

In one example use case, wearable microneedle biosensor devices based on the disclosed technology can be used to enhance the success of a MFTT surgical procedure. As previously discussed, MFTT is a surgical procedure that allows reconstruction of wounds using variable tissue types (e.g., skin, fascia, muscle, and/or bone) through the use of microsurgery to re-sew blood vessels ranging from 1-5 mm in order to autologously transplant that tissue and its blood supply to a distant area on the body for reconstruction. Indications for MFTT include reconstruction following oncologic ablative surgery for advanced tumors that would historically be unresectable, following debridement of non-healing wounds from peripheral vascular disease or prior radiation therapy, or following various traumatic injuries. For the first week after MFTT surgery, the free flap is at risk of failing from venous or arterial compromise (obstruction). Urgent surgical intervention may salvage the flap tissue, but the likelihood of successful salvage decreases when problems are detected late. When a flap totally fails, the result is devastating. The patient must undergo a second reconstruction procedure and there are often delays in care that affect oncologic prognosis. Furthermore, free flap failure results in significant morbidity, expense, and increased length of hospitalization. Thus, early identification of flap failure is an essential prerequisite for flap salvage. The current standard of care typically necessitates a 1-2 day ICU stay where nurses check audible doppler and physical exams hourly. These metrics are qualitative and their sensitivity for detecting early compromise is low.

In some implementations, the wearable microneedle biosensor device can be both continuous and real-time. For example, the device may collect, analyze, and transmit data autonomously. This portable and modular “lab-on-a-chip” multiplexed analysis functionality along with the autonomous specimen collection and data transmission is particularly significant for clinical and translational science goals because the remote-testing aspect could allow investigators access to additional patient populations who might not be able to physically come to the clinic for assessment or specimen collection. Furthermore, to this end, the increased patient convenience of not having to travel, comfort of avoiding multiple collection procedures, and cost savings from the same device giving continuous readings, could empower researchers to ask new questions and generate new hypotheses related to intervention onset or duration. Moreover, the disclosed wearable microneedle biosensor device's ability to quantitatively and directly measure the metabolic status of the tissue, with spatial and temporal information about the tissue, provides new information and capabilities to determine a resolution of measured outcomes along a spectrum between perfectly healthy and completely necrotic. In addition to merely qualitatively identifying transplanted tissue as healthy or unhealthy (i.e., viable from unviable), the disclosed wearable tissue viability sensor technology can be deployed to spatially map regions and subregions of the transplanted tissue along a quantitative viability spectrum (e.g., with respect to concentrations of specific analytes and an assessed score based on those concentration) over one or more periods of time. This viability spectrum resolution and spatial-temporal mapping can empower clinicians in improving patient outcomes for surgical procedures involving tissue transplantation, e.g., including MFTT, as well as researchers to generate new hypotheses and insights into the physiology of tissue viability and lay the foundation for new fields of investigation. Thus, knowledge generated from this device also has the potential to help accelerate the development of new treatment paradigms and new therapeutics to improve human health.

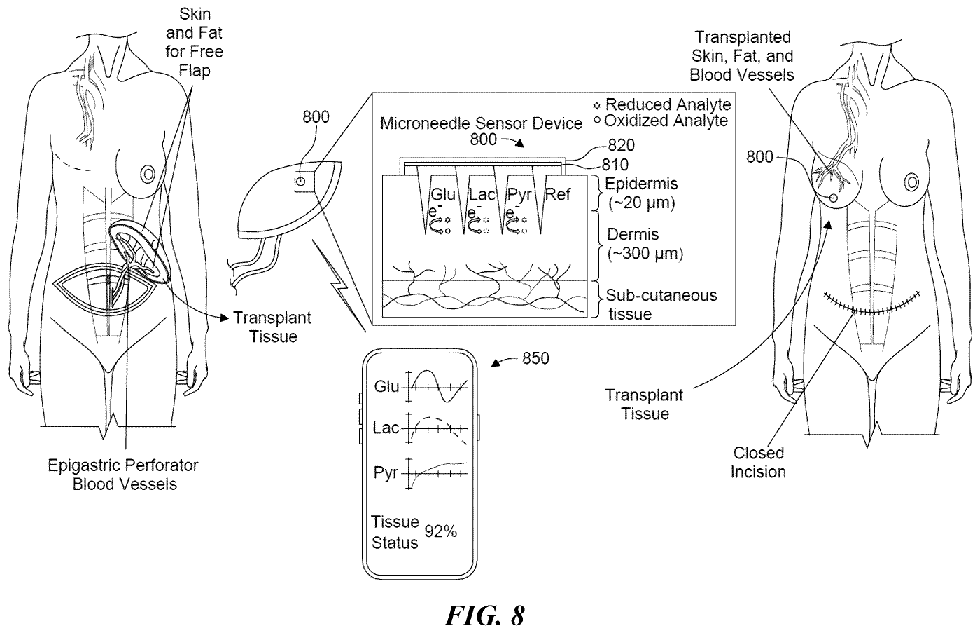

FIG. 8 shows a schematic representation of an example wearable, microneedle sensing device 800, in accordance with the wearable microneedle biosensor device 100 of the present technology, for quantitative, continuous, real-time tissue viability monitoring, particularly for tissue transplantation (e.g., before, during, and after a surgical tissue transplantation procedure). In some implementations, sensors devices based on the disclosed technology (e.g., the sensor device of FIG. 4 and FIG. 7) can be used to improve flap monitoring based on a wireless, wearable microneedle sensing array platform for real-time, continuous simultaneous tracking of multiple metabolic parameters in accordance with the present technology. The example illustration in FIG. 8 shows a microvascular free tissue transfer surgery for breast reconstruction (e.g., deep inferior epigastric artery perforator-based adipofasciocutaneous free flap) following mastectomy. The microneedle sensing device 800 is attached to the transplanted tissue prior to or at the start of the surgery, while the tissue is being transferred, and after the surgery. The zoomed box shows microneedle sensor electrodes of a microneedle sensor unit 810 of the device 800 collecting metabolite levels (e.g., Glucose (Glu), Lactose (Lac), Pyruvate (Pyr)) from the subcutaneous interstitial fluid and wirelessly transmitting the data via wireless communication (e.g., Bluetooth or BLE) between the microneedle sensing device 800 and a remote computing device 850 (e.g., smartphone, tablet, smart wearable device, such as a smartwatch or a smartglasses, or a laptop or desktop computer, or other computer device) for analysis and/or display and/or transfer to a network of computing devices (e.g., cloud computers). The microneedle sensor unit 810 includes a first microneedle sensor electrode configured to detect a first analyte (e.g., glucose), a second microneedle sensor electrode configured to detect a second analyte (e.g., lactate), a third microneedle sensor electrode configured to detect a third analyte (e.g., pyruvate), and a fourth microneedle sensor electrode configured to serve as a reference electrode (e.g., ref).

In some embodiments, for example, the microneedle sensor electrodes of a microneedle sensor unit 810 can be functionalized with a biocatalyst such as an enzyme, e.g., glucose oxidase (GOx), lactate oxidase (LOx), pyruvate oxidase (POx), or other enzyme based on the target analyte or analytes. For example, in some embodiments, the enzyme can be attached to the microneedles sensor electrode via electropolymeric entrapment. The enzyme (e.g., GOx, LOx, POx, other) can be configured to be specific to a target analyte (e.g., glucose, lactate, pyruvate, respectively) present in a biofluid or tissue (e.g., the ISF, or other biofluid for certain applications of the microneedle sensor unit 810, such as blood). In such embodiments, for example, enzyme-based detection of the target analyte, (e.g., glucose, lactate, pyruvate, other) can be performed using the enzyme-functionalized microneedle sensor electrode. In such configurations of the microneedle sensor unit 810, for example, the wearable, microneedle sensing device 800 is able to monitor the current signal of the corresponding hydrogen-peroxide enzymatic reaction product, upon the peroxide oxidation at the corresponding working electrode (e.g., positive potential, with current intensity proportional to the metabolite concentration (e.g., glucose, lactate, pyruvate, other). Other microneedle functionalization techniques can be employed, such as those discussed in U.S. Patent Publication No. 2023/0003725A1 (“Devices and Methods for Aptamer-assisted Microneedle-based Monitoring of Biomarkers”), which is incorporated by reference as part of the disclosure of this patent document for all purposes.

As an example, the microneedle can include an electrode structure in a hollow chamber of the microneedle or protruding on an outside surface of a solid microneedle (e.g., such as a coating), where the electrode structure used for monitoring of the target analyte(s) concentration(s) relies on an immobilized enzyme (e.g., GOx, LOx, POx, other) that facilitates reduction of a produced species (e.g., hydrogen peroxide (H₂O₂)) on the electrode structure. In some embodiments, for example, the electrode structure can include a carbon fiber, carbon paste, or graphite structure, e.g., which can be modified with Prussian blue (PB). For example, the GOx enzyme and PB can be easily incorporated into a carbon paste electrode without any leaching problem while being used for extended periods of time in the fluid matrix.

The microneedle sensor unit 810 is coupled and electrically interfaced with an example embodiment of the electronics unit 120 (depicted in FIG. 8 as electronics unit 820). In some embodiments of the microneedle sensor device 800, the electronics unit 820 can include a case, e.g., to encompass the microneedle sensor unit 810 and electronic components of the electronics unit 820 for shielding from contaminants and preserving the integrity of the electrochemical analyte measurements while the device is coupled with the tissue during the transplantation procedure.

In some example applications, monitoring data from the wearable microneedle sensing device 800 can be congruous with standard monitoring metrics. Among other features and benefits, the wearable microneedle sensing device 800 can augment current methods and may even allow earlier-detection of flap compromise. MFTT is one example of an ideal model system use case because timing of the ischemic insult (e.g., arterial and venous ligation) is precisely known, complete (e.g., 0 to 100% occlusion), and there is a perfectly matched internal control (e.g., the contralateral donor site of the same patient).

In some implementations, a wireless microneedle sensor device or system based on the disclosed technology can be used to perform simultaneous, continuous, and real-time electrochemical monitoring of tissue lactate, glucose, and pyruvate. Preclinical models of microvascular flap ischemia demonstrate that hourly dermal microdialysis can detect lactate and glucose changes, which facilitate early detection of ischemia and discriminate arterial from venous occlusion. Although this microdialysis method was successful on anesthetized pigs, significant challenges prevent its use in humans, including time, cost, and microdialysis catheter placement within the fresh flap. The wireless microneedle sensor device 800 has several distinct advantages over microdialysis techniques. For example, it is engineered such that the microneedle sensor unit 810 also analyzes the data, on-device and in real-time, which reduces cost and is far less labor intensive because it does not require hourly sample collection. Furthermore, it is configured to have a small footprint (e.g., ˜1 cm×1 cm), battery-powered, and can transmit data wirelessly—a considerable advantage for ICU patients whose mobility is already restricted by many lines, wires, and tubes. Additionally, it is capable of continuous, real-time monitoring instead of interval assessment.

Example use cases of the disclosed microneedle sensing devices are presently described. In an example implementation, for example, prior to the in vivo detection on patients and in preclinical models, individual sensors are optimized and characterized for their analytical performance, with particular attention to the sensitivity (dynamic range), selectivity (discrimination against co-existing compounds), and stability in artificial interstitial fluid-like media. The amperometric current output can be correlated with a standard curve of varying known concentrations of the metabolites to obtain concentration readings for samples tested by the sensor.

In one example experimental implementation, standard laboratory rats were employed for study. In such experimental implementations, the distal island groin flap in rats is one example of a well-described surgical model for evaluation of tissue ischemia in MFTT of a fasciocutaneous free flap. The model is based upon a single artery and vein (superficial inferior epigastric artery and vein) that allows for experimenter-controlled initiation and termination of blood flow interruption of the artery or vein for variable durations and simultaneous skin paddle monitoring with a microneedle sensor based on the disclosed technology.

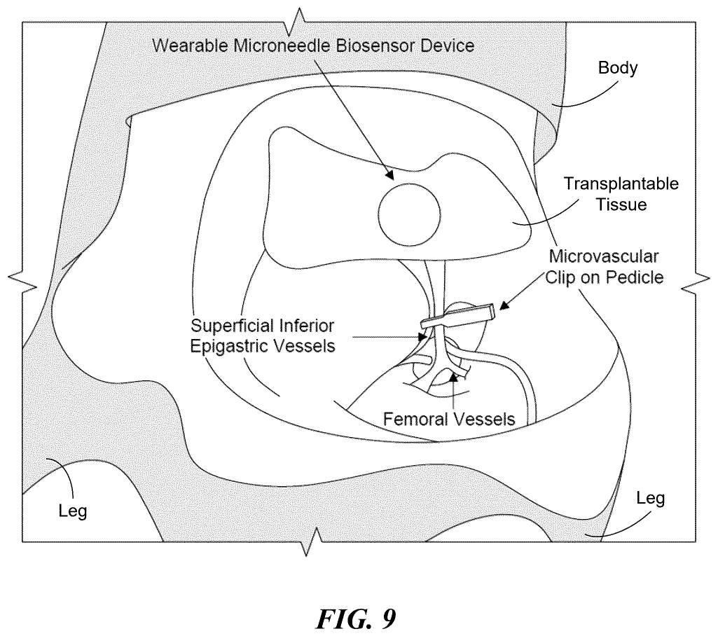

FIG. 9 shows an image of an example surgical implementation of a wearable microneedle biosensor device on an animal model (e.g., rat model), in accordance with the present technology. The example rat model can demonstrate the capability of the wearable microneedle biosensor device to provide continuous, real-time analyte detection in a transplanted tissue of an animal, which includes on-device analyte data processing and wireless transmission to a mobile device receiver (e.g., smartphone). In the example experiments, the rat model implementation protocol includes conducting a tissue transplantation surgery on the animals (e.g., rats), which are to recover from this surgery and be returned to their cages for post op tissue viability monitoring. The protocol includes using the wearable microneedle biosensor device to continuously collect data and to examine the flaps daily for phenotype analysis (% area of flap survival, as determined visually). Following generalized anesthesia, the microneedle sensor device is to be placed on the rat, in accordance with process 210 of the method 200, e.g., a distal island groin flap on a skin paddle that includes the sensor.

Normative data of metabolite levels is to be collected by the test sensor, including the use of an analogous, non-operated skin paddle control sensor, if appropriate. Next, the sensor(s) are to continue collecting metabolite data, e.g., in accordance with process 220 of the method 200, as the experimenter is to place a clamp to completely obstruct blood flow through the flap's proximal artery or vein. Here, a murine free tissue transfer model to test the relative changes in individual metabolite levels (e.g., pyruvate, lactate, and glucose), as measured by the sensor, can produce reliably distinguishable signals that accurately predict whether investigator-initiated single vessel obstruction is venous or arterial in nature. The microneedle sensor is capable of producing a clear and detectable electrical signal of changing metabolite levels, correlated with investigator vessel manipulation that is temporally related and can also predict the nature of the insult (arterial or venous).

According to the example protocol, the clamps are to remain in place for a variable amount of time (e.g., in hours: 0, 2, 4, 6, 8, and permanent) as the sensors collect data while the free flap's skin paddle becomes progressively more ischemic or venous congested. When the clamp(s) are removed, the sensors can continue to collect data as the obstruction is removed and blood flow is restored through the proximal vessels. Depending on how long the clamps were allowed to remain in place, that is, whether clamps were removed before or after the maximal ischemic duration tolerated by the skin paddle (critical ischemia time), the tissue may or may not re-perfuse. If it does not, the tissue would progress to complete necrosis (free flap failure). After analyzing the data, the sensor is able to quantitatively establish a critical threshold of metabolic derangement corresponding to this critical ischemia time, beyond which reperfusion is not expected to occur. In other words, the sensor's data facilitates prediction of the maximal ischemic duration for each flap.

Animals can recover from anesthesia and be returned to their cages with the microneedle sensors in place, which will continue to collect data continuously. The surgical site and flaps can be examined at least daily until animals are to be sacrificed when final phenotype analysis is be determined as percent area of flap survival, which can be established as the gold standard to which metabolite data gathered by the microneedle sensor can be compared.

For power analysis in the initial animal testing, a 40% difference can be assumed between groups with a standard deviation of 10% (beta=0.8, alpha=0.05) resulting in n=3 for each condition (occlusion of artery, vein, or neither) at each time point. These initial experiments can inform power analyses for planning subsequent preclinical experiments. A paired t-test can be used for repeated and contralateral measurements between the sensors over the surgical site (flap tissue) and non-operated side. A p<alpha=0.05 can be considered statistically significant. In some implementations, for example, power consumption can be managed based on managing the frequency of transmitting the data wirelessly from the electronics unit 820 to the remote computing device 850 (e.g., intermittent transmissions of 1 min, 2 min, 5 min, 10 min, or other transmission cadence).

In addition to animal model experimental implementations, clinical detection of tissue metabolite levels using a microneedle sensor device, based on the disclosed technology, intraoperatively during routine MFTT and during the post-operative period following such surgery is described. In an example study, patients older than 18 years of age who are scheduled to undergo microvascular free tissue transfer for reconstruction of a defect resulting from the surgical resection of a tumor were offered the option to participate in this example observational study. The reconstruction includes a skin (cutaneous) component that is external (not entirely within the mouth, pharynx, or other internal surface). Tumors, such as advanced skin cancers, breast cancer, and mucosal cancers of the head and neck, requiring composite resection including skin, among others, would qualify.

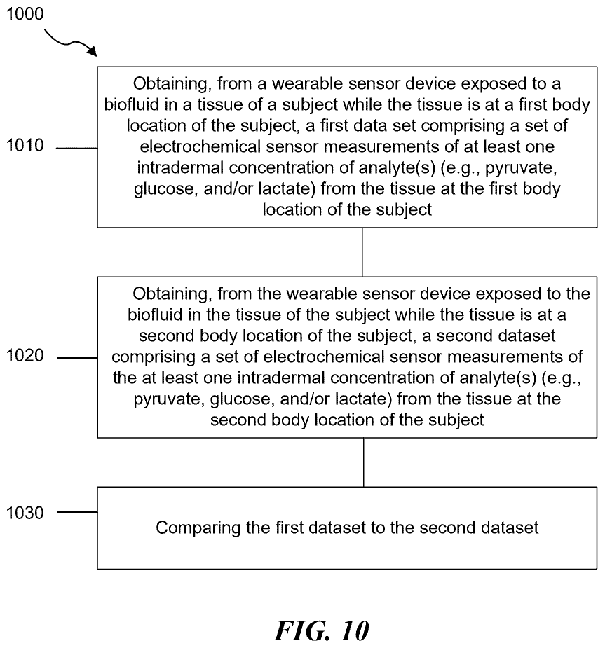

FIG. 10 shows a flow diagram depicting an example method 1000 for assessing tissue viability during a tissue transplantation in accordance with the present technology. The method 1000 includes a process 1010 to obtain, from an example embodiment of the wearable sensor device exposed to a biofluid in a tissue of a subject while the tissue is at a first body location of the subject, a first dataset comprising a set of electrochemical sensor measurements of at least one intradermal concentration of one or more analytes (e.g., pyruvate, glucose, and/or lactate) from the tissue at the first body location of the subject. The method 1000 includes a process 1020 to obtain, from the wearable sensor device exposed to the biofluid in the tissue of the subject, where the tissue has been transplanted to a second body location of the subject, a second dataset comprising a set of electrochemical sensor measurements of the at least one intradermal concentration of one or more analytes (e.g., pyruvate, glucose, and/or lactate) from the tissue at the second body location of the subject. The method 1000 includes a process 1030 to compare the first dataset and the second dataset. In some implementations of the process 1030, comparing the first dataset and the second dataset includes correlating temporal information (e.g., maximums, minimums, changes, rate changes, etc. of the analyte concentration data over time) of the first dataset and the second dataset to temporal stages of the tissue transplantation, e.g., to determine viability of the tissue. In some implementations of the process 1030, the comparing includes determining regional viability of regions of the tissue that was transplanted from the first body location to the second body location. The method 1000 includes a process to obtain, from the wearable sensor device exposed to the biofluid in the tissue of the subject, where the tissue in the process of being transferred from the first body location to the second body location of the subject, a dataset comprising a set of electrochemical sensor measurements of the at least one intradermal concentration of one or more analytes (e.g., pyruvate, glucose, and/or lactate) from the tissue while in transfer and before transplantation at the second body location of the subject. In some implementations of the method 1000, the processes 1010, 1020, and/or 1030 and/or additional processes in accordance with the method 1000 can be repeated.

An example surgical implementation of the method 1000 is presently described. At the time of surgery, one or a plurality of microneedle sensors based on the disclosed technology can be applied to the epidermis to achieve constant access to skin interstitial fluid and secured with a medical-grade bandage along with a wireless circuit board. For example, one (or more) sensor(s) can be placed on the skin of tissue that is incorporated within the design for the free flap, and a second sensor, e.g., as a baseline or control sensor, can be placed nearby on adjacent, non-free flap skin. Skin adjacent to the free flap donor site may be chosen here because it has similar epidermal and dermal thickness and tissue quality compared to other sites on the body. Alternatively, the second sensor can be applied to the corresponding contralateral region (e.g., if a left anterolateral thigh free flap is planned, the second sensor can be applied to the skin overlying the non-transferred right thigh). Both continuous and one-time periodic measurements can be carried out wirelessly using the microneedle sensors.

As an example experimental implementation of the method 1000, a test can involve continuously and minimally-invasively measuring the target metabolite levels in the interstitial fluid in accordance with the processes 1010 and 1020 using the microneedle-based platform for the duration of the patient's surgery, e.g., including prior to transplantation, during transplantation, and after transplantation. Typical microvascular free tissue transfer surgical cases last 7-12 hours, for example, and includes free flap harvest (e.g., a period typically lasting 1-6 hours where both the planned free flap tissue and adjacent non-transferred tissue have baseline perfusion/blood flow), free flap inset and microvascular anastomosis (e.g., a period typically lasting 2-3 hours where the blood vessels perfusing the free flap are disconnected from the donor site and the free flap is completely ischemic but the adjacent non-transferred tissue has baseline perfusion), and wound closure (e.g., a period typically lasting 1-3 hours where the adjacent non-transferred tissue has baseline perfusion/blood flow, and the transferred free flap tissue is re-perfused by blood vessels from the recipient site). The natural flow of surgery produces conditions to test the exemplary microneedle sensor device as metabolites measured from the transferred free tissue flap can be measured at baseline, during tissue ischemia, and then during reperfusion following microvascular anastomosis. Each of these stages can be compared to an adjacent non-transferred tissue site (e.g., the second sensor) as an ideal control that would eliminate confounding clinical scenarios, such as hypovolemia (where intravascular resuscitation is insufficient and mild tissue ischemia may be detected systemically). The stability of such continuous sensing operation can be evaluated throughout each case.

In some implementations of the method 1000, following the intraoperative period above, data can continue to be collected from the microneedle sensor devices post-operatively in accordance with the processes 1010 and 1020. Both continuous and one-time periodic measurements can be carried out wirelessly using the microneedle sensors. The method may involve continuously measuring the target metabolite levels in the interstitial fluid in a non-invasive fashion using the microneedle-based platform for the duration of the patient's hospitalization until the standard of care flap checks are discontinued as clinically indicated. Data obtained from the first sensor(s) on the free flap can be compared to that from the second sensor from the donor site as an ideal control that would eliminate confounding clinical scenarios, such as hypovolemia (where intravascular resuscitation is insufficient and mild tissue ischemia may be detected systemically) or hyperglycemia (glucocorticoid steroids are sometimes given after this surgery and they can elevate blood glucose levels systemically). Stability of continuous sensing can be evaluated throughout each case.

Data measured as described above can be converted to metabolite concentration readings via comparison to standard curves from lab-based metabolite measurements as previously discussed. In accordance with the process 1030, trends in metabolite concentrations over time may be correlated to the stages of surgery including start of free flap ischemia (when the blood vessels from the donor site feeding the flap are cut) and start of free flap reperfusion (when the flap is connected to the new blood vessels at the reconstruction site) and calculate the latency to detectable signal compared to the signal from the microneedle sensor device on the adjacent non-transferred tissue. For example, when the surgical transplantation is successful (i.e., the transplanted tissue is viable), both the flap sensor(s) and adjacent, non-transferred sensor are expected to be similar until the time of ischemia. Once the blood vessel pedicle perfusing the flap is cut, the time until the two sensors produce a signal difference for each metabolite that is statistically significant can be measured (e.g., the adjacent tissue glucose, pyruvate, and lactate levels may remain stable while flap glucose and pyruvate would decrease and flap lactate would increase-however, these metabolic changes, which are driven by glycolysis and the lack of fresh blood bringing glucose and pyruvate and venous outflow recycling the lactate waste, would not occur until the tissue starts to become ischemic).

Data analysis can be performed similarly to that described above in connection to the intraoperative period. For instance, the data obtained from our microneedle sensing device can be compared and correlated to the examining provider's overall assessment of free flap tissue health. If any free flap tissue compromise occurs, the performance of the system can be assessed against the standard of care monitoring methods and intraoperative findings if the case requires surgical intervention. For example, if a clot forms in the free flap pedicle's vein it will cause venous obstruction without arterial insufficiency (because the artery inflow of blood is normal) which might produce one signal pattern, while if a clot forms in the artery it will cause arterial insufficiency without venous obstruction and produce a distinct signal pattern. If either of these complications occur and the patient is brought back to the operating room, the surgeon could discover a cause by physically examining the blood vessels.

In the presently described example study, the sensor readouts are configured to be precise, accurate, and repeatable in patients since the initial chosen metabolites (e.g., glucose, pyruvate, and lactate) were selected based on prior clinical trials employing microdialysis measuring the same metabolites. Alternative strategies would be changing the location of the sensor on the flap's skin paddle with respect to the precise location of perforating vessels (e.g., closer or further to capture the metabolite concentration from the center or periphery of that vascular territory), placing multiple sensors at different locations on the same flap, or modifying design or fabrication details of the microneedle sensor to optimize it for this particular indication.

For MFTT of the head and neck, for example, the rate of free flap failure in which critical tissue ischemia results in a non-viable flap is about 3-5%, and the rate of threatened flap ischemia requiring a return to the operating room for assessment or revision of the microvascular anastomosis is about 15-30%. Therefore, for example, implementing the surgical procedure in 35 patients would likely produce 1-3 cases of flap failure and 5-11 cases of threatened flap ischemia. To achieve 80% sensitivity and 80% specificity of the example transplantable tissue viability microneedle sensor device in predicting free flap vessel compromise and/or reoperation, a sample size of n=35 was calculated, assuming a precision of 25%, confidence level of 95% (p<alpha=0.05), and an expected dropout rate of 5%. Differences between sexes are not anticipated, and enough of samples of each sex can be gathered to make a preliminary comparison.

The disclosed wearable microneedle biosensor devices, systems, and methods have the potential to be massively impactful for both clinical care and scientific investigation across many medical fields. The disclosed embodiments provide a tool that allows new questions to be asked, as well as a platform technology that is modular and can be tailored to further extend applicability. Among other features and benefits, the disclosed embodiments address the challenge of tissue viability assessment, as outlined above, and the advancement of public health and healthcare goals that follow. Wearable, wireless, multiplexed microneedle-based biosensors, as described in this patent document, offer a modular platform technology whose target analytes can be substituted to address a staggeringly wide array of conditions.

### EXAMPLES

In some embodiments in accordance with the present technology (example A1), a method for assessing tissue viability during a tissue transplantation includes attaching a wearable sensor device comprising at least one microneedle having an electrochemical sensor electrode to detect an electrical signal from a reaction with a target analyte in a biofluid exposed to the at least one microneedle, wherein the wearable sensor device is attached to a tissue of an anatomic structure of a subject at an outer surface of the anatomic structure while at a first body location of the subject; measuring a first intradermal concentration of at least one metabolite from the tissue at the first body location of the subject; and detaching at least a portion of the anatomic structure that includes the tissue with the attached wearable sensor device, thereby forming a detached body part with the wearable sensor device attached to the outer surface of the detached body part; attaching the detached body part at a second body location of the subject; and measuring a second intradermal concentration of the at least one metabolite from the tissue at the second body location of the subject.

Example A2 includes the method of example A1 or any of examples A1-A8, wherein measurements of the first and the second intradermal concentrations are measured continuously during when the tissue is at the first body location and during when the tissue is attached to the second body location.

Example A3 includes the method of example A1 or example A2 or any of examples A1-A8, comprising measuring a third intradermal concentration of the at least one metabolite from the tissue between the detaching the at least a portion of the anatomic structure from the first body location of the subject and the attaching the detached body part at the second body location of the subject.

Example A4 includes the method of example A3 or any of examples A1-A8, wherein measurements of the first, the second, and the third intradermal concentrations are measured continuously during the detaching of the at least a portion of the anatomic structure from the first body location of the subject and the attaching of the detached body part at the second body location of the subject.

Example A5 includes the method of any of examples A1-A4 or any of examples A1-A8, wherein the wearable sensor device comprises: a substrate comprising an electrically insulative material, the at least one microneedle disposed on the substrate and comprising a body region and a tip region, and a chemical layer on the tip region of the at least one microneedle configured to interact with the target analyte in the biofluid and produce the electrical signal at the at least one electrochemical sensor electrode.

Example A6 includes the method of example A5 or any of examples A1-A8, wherein the at least one microneedle is a hollow microneedle structure.

Example A7 includes the method of example A5 or any of examples A1-A8, wherein the at least one microneedle is a solid microneedle structure.

Example A8 includes the method of example A7 any examples A1-A6, wherein the wearable sensor device comprises an array of microneedles comprising the at least one microneedle.

In some embodiments in accordance with the present technology (example B1), a method for assessing tissue viability during a tissue transplantation includes attaching a wearable sensor device comprising at least one microneedle having an electrochemical sensor electrode to detect an electrical signal from a reaction with a target analyte in a biofluid exposed to the at least one microneedle, wherein the wearable sensor device is attached to a tissue of an anatomic structure of a subject at an outer surface of the anatomic structure while at a first body location of the subject; measuring a first intradermal concentration of at least one metabolite from the tissue at the first body location of the subject; and detaching at least a portion of the anatomic structure that includes the tissue with the attached wearable sensor device, thereby forming a detached body part with the wearable sensor device attached to the outer surface of the detached body part; attaching the detached body part at a second body location of the subject; and measuring a second intradermal concentration of the at least one metabolite from the tissue at the second body location of the subject.

Example B2 includes the method of example B1 or any of examples B1-B8, wherein measurements of the first and the second intradermal concentrations are measured continuously during when the tissue is at the first body location and during when the tissue is attached to the second body location.

Example B3 includes the method of example B1 or any of examples B1-B8, further including measuring a third intradermal concentration of the at least one metabolite from the tissue between the detaching the at least a portion of the anatomic structure from the first body location of the subject and the attaching the detached body part at the second body location of the subject.

Example B4 includes the method of example B3 or any of examples B1-B8, wherein measurements of the first, the second, and the third intradermal concentrations are measured continuously during the detaching of the at least a portion of the anatomic structure from the first body location of the subject and the attaching of the detached body part at the second body location of the subject.

Example B5 includes the method of example B1 or any of examples B1-B8, wherein the wearable sensor device includes a substrate comprising an electrically insulative material, the at least one microneedle disposed on the substrate and comprising a body region and a tip region, and a chemical layer on the tip region of the at least one microneedle configured to interact with the target analyte in the biofluid and produce the electrical signal at the at least one electrochemical sensor electrode.

Example B6 includes the method of example B5 or any of examples B1-B8, wherein the at least one microneedle is a hollow microneedle structure, or wherein the at least one microneedle is a solid microneedle structure.

Example B7 includes the method of example B1 or any of examples B1-B8, wherein the wearable sensor device comprises an array of microneedles comprising a plurality of the at least one microneedle.

Example B8 includes the method of example B7 or any of examples B1-B6, wherein the plurality of the at least one microneedle are distributed in multiple regions of the tissue, and wherein the method further includes analyzing the first intradermal concentration and the second intradermal concentration measured from the plurality of the at least one microneedle to produce a spatial-temporal map of viability of the tissue across the multiple regions of the tissue and over a period of time.

In some embodiments in accordance with the present technology (example B9), a method for assessing tissue viability during a tissue transplantation includes obtaining, from a wearable sensor device exposed to a biofluid in a tissue of a subject while the tissue is at a first body location of the subject, a first dataset comprising at least one intradermal concentration of one or more analytes from the tissue at the first body location of the subject; obtaining, from the wearable sensor device exposed to the biofluid in the tissue of the subject while the tissue is at a second body location of the subject, a second dataset comprising at least one intradermal concentration of the one or more analytes from the tissue at the second body location of the subject, wherein, prior to obtaining the second data set, the tissue was detected from the first body location and transplanted at the second body location of the subject; and comparing, by a data processing unit configured to process data obtained from the wearable sensor device, the first dataset to the second dataset, wherein the comparing includes correlating temporal information in the first dataset and the second dataset to temporal stages of the tissue transplantation, wherein the at least one intradermal concentration of the one or more analytes at the first body location of the subject and the at least one intradermal concentration of the one or more analytes at the second body location of the subject are measured by one or more microneedle electrochemical sensor electrodes of the wearable sensor device.

Example B10 includes the method of example B9 or any of examples B9-B22, wherein the one or more analytes include at least one of pyruvate, glucose, or lactate.

Example B11 includes the method of example B9 or any of examples B9-B22, wherein the one or more analytes include a plurality of the analytes comprising pyruvate, glucose, and lactate.

Example B12 includes the method of example B9 or any of examples B9-B22, wherein the one or more microneedle electrochemical sensor electrodes include a hollow microneedle structure, or wherein one or more microneedle electrochemical sensor electrodes include a solid microneedle structure.

Example B13 includes the method of example B9 or any of examples B9-B22, wherein at least one of the one or more microneedle electrochemical sensor electrodes comprises a chemical layer deposited on a tip region of an electrode surface or coated on an outer wall of a microneedle surface, wherein the chemical layer is configured to chemically react with an analyte of the one or more analytes thereby facilitating electrochemical detection of a reaction involving the chemical layer and the analyte.

Example B14 includes the method of example B9 or any of examples B9-B22, wherein the wearable sensor device includes an electronics unit configured to wirelessly output data corresponding measurements of the at least one intradermal concentration measured by one or more microneedle electrochemical sensor electrodes of the wearable sensor device to the data processing unit configured on a remote computing device.

Example B15 includes the method of example B14 or any of examples B9-B22, wherein the remote computing device includes a smartphone, a tablet, a smart wearable device, a laptop computer, or desktop computer.

Example B16 includes the method of example B9 or any of examples B9-B22, further including determining a viability of the tissue based at least in part on the comparing the first dataset to the second dataset.

Example B17 includes the method of example B9 or any of examples B9-B22, further including determining a viability of the tissue based at least in part on calculating a signal difference between (i) a first set of measurements obtained at a first time or time period from the wearable sensor device at the first body location and the second body location and (ii) a second set of measurements obtained at a second time or time period from another wearable sensor device attached to another, non-transplanted tissue of the subject at a third body location of the subject, wherein the first set of measurements and the second set of measurements indicate concentrations of the one or more analytes.

Example B18 includes the method of example B17 or any of examples B9-B22, wherein the method includes determining statistical significance of the signal difference based on a time difference between the first time and the second time.

Example B19 includes the method of example B9 or any of examples B9-B22, wherein the wearable sensor device is attached subcutaneously to the tissue, and wherein the biofluid is skin interstitial fluid.

Example B20 includes the method of example B9 or any of examples B9-B22, wherein the wearable sensor device comprises an array of microneedle electrochemical sensor electrodes.

Example B21 includes the method of example B9 or any of examples B9-B22, wherein the method comprises implementing a plurality of the wearable sensor devices to obtain the first dataset and the second dataset, where the plurality of the wearable sensor devices are distributed in multiple regions of the tissue, and wherein the method further comprises analyzing tissue viability at the multiple regions of the tissue based on spatial information associated with the first dataset and the second dataset measured from the plurality of the wearable sensor devices to produce a spatial-temporal map of viability of the tissue across the multiple regions of the tissue and over a period of time.

Example B22 includes the method of example B21 or any of examples B9-B20, wherein at least some of the plurality of wearable sensor devices are attached to a shared substrate.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for assessing tissue viability during a tissue transplantation, the method comprising:
attaching a wearable sensor device comprising at least one microneedle having an electrochemical sensor electrode to detect an electrical signal from a reaction with a target analyte in a biofluid exposed to the at least one microneedle, wherein the wearable sensor device is attached to a tissue of an anatomic structure of a subject at an outer surface of the anatomic structure while at a first body location of the subject;
measuring a first intradermal concentration of at least one metabolite from the tissue at the first body location of the subject; and
detaching at least a portion of the anatomic structure that includes the tissue with the attached wearable sensor device, thereby forming a detached body part with the wearable sensor device attached to the outer surface of the detached body part;
attaching the detached body part at a second body location of the subject; and
measuring a second intradermal concentration of the at least one metabolite from the tissue at the second body location of the subject.

2. The method of claim 1, wherein measurements of the first and the second intradermal concentrations are measured continuously during when the tissue is at the first body location and during when the tissue is attached to the second body location.

3. The method of claim 1, further comprising:
measuring a third intradermal concentration of the at least one metabolite from the tissue between the detaching the at least a portion of the anatomic structure from the first body location of the subject and the attaching the detached body part at the second body location of the subject.

4. The method of claim 3, wherein measurements of the first, the second, and the third intradermal concentrations are measured continuously during the detaching of the at least a portion of the anatomic structure from the first body location of the subject and the attaching of the detached body part at the second body location of the subject.

5. The method of claim 1, wherein the wearable sensor device comprises:
a substrate comprising an electrically insulative material,
the at least one microneedle disposed on the substrate and comprising a body region and a tip region, and
a chemical layer on the tip region of the at least one microneedle configured to interact with the target analyte in the biofluid and produce the electrical signal at the at least one electrochemical sensor electrode.

6. The method of claim 5, wherein the at least one microneedle is a hollow microneedle structure, or wherein the at least one microneedle is a solid microneedle structure.

7. The method of claim 1, wherein the wearable sensor device comprises an array of microneedles comprising a plurality of the at least one microneedle.

8. The method of claim 7, wherein the plurality of the at least one microneedle are distributed in multiple regions of the tissue, and wherein the method further comprises:
analyzing the first intradermal concentration and the second intradermal concentration measured from the plurality of the at least one microneedle to produce a spatial-temporal map of viability of the tissue across the multiple regions of the tissue and over a period of time.

9. A method for assessing tissue viability during a tissue transplantation, the method comprising:
obtaining, from a wearable sensor device exposed to a biofluid in a tissue of a subject while the tissue is at a first body location of the subject, a first dataset comprising at least one intradermal concentration of one or more analytes from the tissue at the first body location of the subject;
obtaining, from the wearable sensor device exposed to the biofluid in the tissue of the subject while the tissue is at a second body location of the subject, a second dataset comprising at least one intradermal concentration of the one or more analytes from the tissue at the second body location of the subject, wherein, prior to obtaining the second data set, the tissue was detected from the first body location and transplanted at the second body location of the subject; and
comparing, by a data processing unit configured to process data obtained from the wearable sensor device, the first dataset to the second dataset, wherein the comparing includes correlating temporal information in the first dataset and the second dataset to temporal stages of the tissue transplantation,
wherein the at least one intradermal concentration of the one or more analytes at the first body location of the subject and the at least one intradermal concentration of the one or more analytes at the second body location of the subject are measured by one or more microneedle electrochemical sensor electrodes of the wearable sensor device.

10. The method of claim 9, wherein the one or more analytes include at least one of pyruvate, glucose, or lactate.

11. The method of claim 9, wherein the one or more analytes include a plurality of the analytes comprising pyruvate, glucose, and lactate.

12. The method of claim 9, wherein the one or more microneedle electrochemical sensor electrodes include a hollow microneedle structure, or wherein one or more microneedle electrochemical sensor electrodes include a solid microneedle structure.

13. The method of claim 9, wherein at least one of the one or more microneedle electrochemical sensor electrodes comprises a chemical layer deposited on a tip region of an electrode surface or coated on an outer wall of a microneedle surface, wherein the chemical layer is configured to chemically react with an analyte of the one or more analytes thereby facilitating electrochemical detection of a reaction involving the chemical layer and the analyte.

14. The method of claim 9, wherein the wearable sensor device includes an electronics unit configured to wirelessly output data corresponding measurements of the at least one intradermal concentration measured by one or more microneedle electrochemical sensor electrodes of the wearable sensor device to the data processing unit configured on a remote computing device.

15. The method of claim 14, wherein the remote computing device includes a smartphone, a tablet, a smart wearable device, a laptop computer, or desktop computer.

16. The method of claim 9, further comprising:
determining a viability of the tissue based at least in part on the comparing the first dataset to the second dataset.

17. The method of claim 9, further comprising:
determining a viability of the tissue based at least in part on calculating a signal difference between (i) a first set of measurements obtained at a first time or time period from the wearable sensor device at the first body location and the second body location and (ii) a second set of measurements obtained at a second time or time period from another wearable sensor device attached to another, non-transplanted tissue of the subject at a third body location of the subject, wherein the first set of measurements and the second set of measurements indicate concentrations of the one or more analytes.

18. The method of claim 17, comprising:
determining statistical significance of the signal difference based on a time difference between the first time and the second time.

19. The method of claim 9, wherein the wearable sensor device is attached subcutaneously to the tissue, and wherein the biofluid is skin interstitial fluid.

20. The method of claim 9, wherein the wearable sensor device comprises an array of microneedle electrochemical sensor electrodes.

21. The method of claim 9, wherein the method comprises implementing a plurality of the wearable sensor devices to obtain the first dataset and the second dataset, where the plurality of the wearable sensor devices are distributed in multiple regions of the tissue, and wherein the method further comprises:
analyzing tissue viability at the multiple regions of the tissue based on spatial information associated with the first dataset and the second dataset measured from the plurality of the wearable sensor devices to produce a spatial-temporal map of viability of the tissue across the multiple regions of the tissue and over a period of time.

22. The method of claim 21, wherein at least some of the plurality of wearable sensor devices are attached to a shared substrate.
